(19) United States
(12) Plant Patent Application Publication
(10) Pub. No.: US 2015/0201541 P1
(43) Pub. Date: Jul. 16, 2015

(54) Guzmania Plant Named 'G9191'

(50) ;

(71) Applicant: Bjorn Bunnik, Kudelstaart (NL)

(72) Inventor: Bjorn Bunnik, Kudelstaart (NL)

(21) Appl. No.: 13/999,082

(22) Filed: Jan. 10, 2014

(51) Int. Cl. A01H 5/00

(52) U.S. Cl. PLT/371

(57) ABSTRACT

A new and distinct Guzmania cultivar named ‘G9191’ is disclosed, characterized by unique pink/purple inflorescence color, green leaves and large inflorescence. The new variety is a Guzmania, typically produced as an indoor ornamental plant.

### LATIN NAME OF THE GENUS AND SPECIES

Guzmania hybrid

### VARIETY DENOMINATION

‘G9191’

### BACKGROUND OF THE INVENTION

This application relates to a new cultivar of Guzmania. The new variety is the product of chance discovery, by the inventor, Bjorn Bunnik. The new variety originated as a naturally occurring whole plant mutation from the unpatented commercial variety Guzmania ‘Voila’.

The new variety was first selected by the inventor, in at a commercial greenhouse in Kudelstaart, the Netherlands. After identifying the new variety as a potentially interesting selection, the inventor continued confidential testing and propagation of ‘G9191’, assessing stability of the unique characteristics of this variety.

Asexual reproduction of the new cultivar ‘G9191’ was first performed at a commercial laboratory in Kudelstaart, Netherlands by tissue culture in 2008. Access to all plants was restricted, as plants were kept in a greenhouse or laboratory not open to the public. Through subsequent propagation, multiple generations have been reproduced, which have shown that the unique features of this cultivar are stable and reproduced true to type.

### SUMMARY OF THE INVENTION

The cultivar ‘G9191’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘G9191.’ These characteristics in combination distinguish ‘G9191’ as a new and distinct Guzmania cultivar:

1. Pink-purple inflorescence.

2. Green leaves.

3. Large inflorescence.

### PARENT COMPARISON

Plants of the new cultivar ‘G9191’ are similar to the parent in most horticultural characteristics. However the new variety, ‘G9191’ differs from the ‘Voila’ in the following characteristics:

1. New variety is a more compact plant.

2. Larger inflorescence.

3. Shorter inflorescence.

### COMMERCIAL COMPARISON

‘G9191’ is similar in most horticultural characteristics to the unpatented commercial variety Guzmania ‘Victory’ in most horticultural characteristics. However the new variety, ‘G9191’ differs from ‘Bolero’ in the following characteristics:

1. Larger inflorescence.

2. Larger plant size.

### BRIEF DESCRIPTION OF THE PHOTOGRAPH

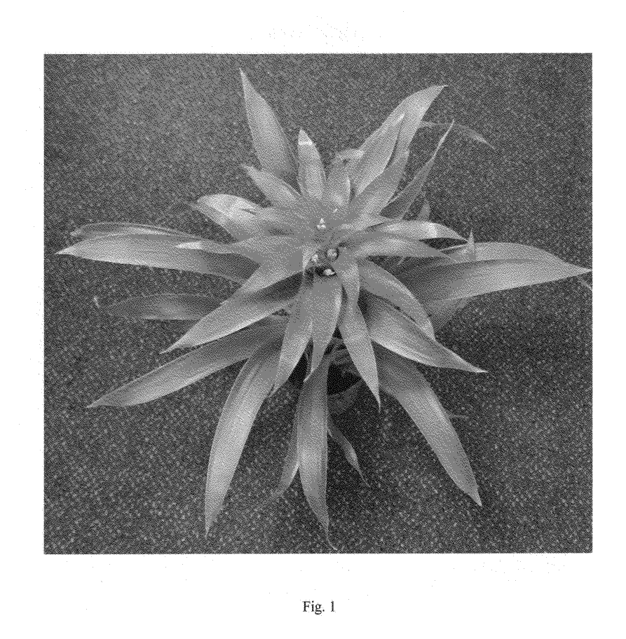

The accompanying photograph in FIG. 1 illustrates in full color a typical plant of ‘G9191’ grown in a commercial greenhouse in De Kwakel, Netherlands. The plant is 20 months old from a rooted cutting, in a 12 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe a typical plant of ‘G9191’ grown in a commercial greenhouse in De Kwakel, Netherlands. The plant is 20 months old from a rooted cutting, in a 12 cm pot. Temperatures ranged between 20° C. and 28° C. during the day 18° C. and 22° C. at night. No supplemental lighting was given. Measurements and numerical values represent averages of typical plant types.
Botanical classification: Guzmania hybrid ‘G9191’
Propagation:
   Type of propagation typically used.—Tissue culture.
   Time to initiate roots.—About 15 days at approximately 20° C.
   Time to produce a rooted cutting/liner.—About 180 days at approximately 22° C.
   Root description.—Moderately dense, moderately branched, fine, fibrous, not fleshy, coloured brown; near RHS 200A to 200D.
Plant:
   Growth habit.—Upright, monopodial.
   Height.—Average: 38.7 cm
   Plant spread.—Average: 53.1 cm.
   Normal pot size.—12 cm (circular).
   Growth rate.—Moderate.
   Branching characteristics.—Monopodial, leaves form a basal rosette, inflorescence grows from the center of the rosette.
   Number of leaves per scape.—Average: 25.
   Stem length.—No stems present, scape grows directly from rosette.
Foliage:
   Leaf.—Arrangement: Rosulate; leaves (average: 25) placed in a basal rosette Average Length: Approximately 36.4 cm. Average Width: Approximately 3.7 cm (measured at 50% of the leaf blade) Shape of blade: Ligulate Aspect: Base in an average angle of 45° to horizontal, tip arching downward in an average angle of 20° to horizontal Apex: Apiculate, curved downward in an average angle of 20° to horizontal Base: Sheathing Sheath length: Average 8.0 cm Sheath width: Average 7.4 cm. Sheath color: Greyed-yellow, near RHS 161 A Margin: Entire Texture of top surface: Glabrous, glossy Texture of bottom surface: Glabrous, glossy Color: Young foliage upper side: Green; near RHS 143A. Young foliage under side: Green to yellow-green; in between near RHS 143A and 146A. Mature foliage upper side: Green; near RHS 143A, but slightly darker. Mature foliage under side: Green to yellow-green; in between near RHS 143A and 146A. Venation: Type: Parallel Venation coloration: As leaf blade; upper side green; near RHS 143A but slightly darker, under side green to yellow-green; in between near RHS 143A and 146A.
   Petiole.—No petioles present, leaves sheathing.
Inflorescence:
   General description.—Scape growing from the centre of the basal rosette, inflorescence; only bracts visible, individual flowers not observed. Inflorescence Height, excluding scape: Approximately 10.9 cm. Inflorescence Width: Approximately 27.8 cm.
   Scape.—Length: Approximately 22.5 cm. Width: Approximately 1.1 cm. Aspect: Approximately 90° angle to rosette (near vertical). Strength: Very strong Color: Green-white, near RHS 157D.
   Bracts.—.
   Scape/lowermost bracts.—Shape: Ligulate Length: Approximately 22.1 m (excluding sheath). Width: Approximately 3.3 cm (measured at 50% of the leaf blade) Quantity: Average 7 Tip: long apiculate Base: Sheathing. Sheath length: Approximately 5.4 cm Sheath width: Approximately 5.8 cm. Sheath color: Yellow-green to greyed-yellow; in between near RHS 152D and 160A. Margin: Entire Color upper side: Green to yellow-green; in between near RHS 143A and 146A, lower half slightly (lower bracts) to very strongly (upper bracts) tinged red-purple; near RHS 58B, especially at the margins. Color under side: Yellow-green; near RHS 146B, darker towards the base; near RHS 148A, lower half slightly (lower bracts) to very strongly (upper bracts) tinged red-purple; near RHS 58A, especially at the margins.
   Terminal bracts.—Shape: Ligulate Length: Approximately 9.9 cm (incl. sheath). Width: Approximately 2.6 cm (measured at 50% of the leaf blade) Quantity: Average 19 Tip: Apiculate to long apiculate Base: Sheathing Sheath length: Approximately 3.7 cm. Sheath width: Approximately 5.8 cm. Sheath color: Upper side greyed-yellow; near RHS 161C, under side yellow-green; near RHS 151 A, strongly tinged greyed-orange; in between near RHS 163A and 163B, especially in the centre of the sheath. Margin: Entire Coloration Terminal Bracts: Upper side: Red-purple; near RHS 61B, top of lower terminal bracts green to yellow-green; in between near 143A and 146A. Under side: Red-purple; near RHS 61B, top of lower terminal bracts yellow-green; in between near 146B.
Flower: Individual Flowers not observed.
Reproductive organs: Not observed
Other characteristics:
   Disease resistance.—Neither resistance nor susceptibility to the normal diseases and pests of Guzmania has been observed.
   Temperature tolerance.—Normal.
   Fruit/seed production.—No fruits/seeds detected to date.

What is claimed is:

1. A new and distinct cultivar of Guzmania plant named ‘G9191’ as herein illustrated and described.
